(19) United States
(12) Patent Application Publication
(10) Pub. No.: US 2025/0145556 A1
(43) Pub. Date: May 8, 2025

(54) STORAGE AND/OR TRANSPORT OF ETHYLENICALLY UNSATURATED COMPOUNDS

(71) Applicant: BASF SE, Ludwigshafen am Rhein (DE)

(72) Inventors: Jan Niclas GORGES, Ludwigshafen am Rhein (DE); Juergen ROMEIS, Ludwigshafen am Rhein (DE); Friederike FLEISCHHAKER, Ludwigshafen am Rhein (DE); Rebecca SURE, Ludwigshafen am Rhein (DE); Friedrich-Georg MARTIN, Ludwigshafen am Rhein (DE); Christian REIN, Ludwigshafen am Rhein (DE)

(21) Appl. No.: 18/832,991

(22) PCT Filed: Jan. 18, 2023
(86) PCT No.: PCT/EP2023/051052

§ 371 Date: Jul. 25, 2024

(30) Foreign Application Priority Data
Jan. 28, 2022 (EP) 22153813.5

(51) Int. Cl. C07C 51/50; C08F 2/40; C08F 118/14

(52) U.S. Cl. CPC C07C 51/50; C08F 2/40; C08F 118/14; C08F 2438/00

(57) ABSTRACT

The present invention relates to a method of storage and/or transport of an ethylenically unsaturated compound, wherein the ethylenically unsaturated compound is protected against unwanted free-radical polymerization with a phenol.

The present invention relates to a method of storage and/or transport of an ethylenically unsaturated compound, wherein the ethylenically unsaturated compound is protected against unwanted free-radical polymerization with a phenol.

Chemical compounds having one or more ethylenically unsaturated groups have a pronounced tendency to free-radical polymerization. Such compounds are therefore also referred to hereinafter as polymerizable compounds. The tendency of these compounds to free-radical polymerization means that they are used as monomers for the production of polymers. However, the pronounced tendency to free-radical polymerization of these compounds is also a disadvantage in that undesired spontaneous free-radical polymerization can occur during storage and transport and also during chemical and physical processing such as distillation or rectification, particularly under the action of energy such as heat and/or light. Such uncontrolled polymerizations can result in the gradual formation of polymer deposits, for example on heated surfaces, that necessitates removal of the polymer deposits and thus often results in shortened operating times. The uncontrolled polymerizations may even proceed explosively.

During storage and transport and also during chemical and physical processing of ethylenically unsaturated compounds that have a tendency to free-radical polymerization or mixtures that comprise such compounds, it is therefore customary to add compounds that prevent or at least retard undesired spontaneous free-radical polymerization. Such substances are often referred to as polymerization inhibitors.

Polymerization inhibitors may be employed as individual chemical compounds or as mixtures of compounds. Particular requirements are placed on the polymerization inhibitor, depending on its field of use. For a polymerization inhibitor to be suitable as a transport and/or storage stabilizer of ethylenically unsaturated compounds it is important that the efficiency of the polymerization inhibitor, i.e. the extent of inhibition of polymerization, is controllable. Under the conditions of storage and/or transport of the ethylenically unsaturated compounds, the polymerization inhibitor should adequately prevent or retard undesired spontaneous free-radical polymerization, whereas the desired free-radical polymerization of the ethylenically unsaturated compounds should be possible under appropriate polymerization conditions without the need to first have to separate the polymerization inhibitor used during storage and/or transport. If the stabilizer used during storage and/or transport is not separated for the desired polymerization, it is important that it does not adversely affect the desired polymerization, for example unintentionally act as a chain-transfer agent.

In terms of global production volume, acrylic acid is without doubt one of the most important ethylenically unsaturated compounds. During storage and/or transport, acrylic acid is normally stabilized against undesired spontaneous free-radical polymerization with 0.018% to 0.022% by weight of hydroquinone monomethyl ether (MEHQ), based on the amount of acrylic acid. For adequate stabilization of acrylic acid against undesired spontaneous free-radical polymerization using MEHQ, it is necessary that oxygen is dissolved in the acrylic acid in sufficient amounts. Sufficient amounts of oxygen are generally dissolved in the acrylic acid when acrylic acid is stored and/or transported in an atmosphere containing 5% to 21% by volume of oxygen. In the desired polymerization of acrylic acid the content of dissolved oxygen in the acrylic acid is decreased, thus reducing the efficiency of MEHQ as a polymerization inhibitor enough for acrylic acid to undergo polymerization in the presence of MEHQ.

In addition to use as a polymerization inhibitor for the storage and/or transport of acrylic acid, MEHQ is also used as a polymerization inhibitor for the storage and/or transport of methacrylic acid, acrylic esters and/or methacrylic esters or of mixtures comprising one or more of said compounds.

Its widespread use makes MEHQ one of the most important storage and/or transport stabilizers for ethylenically unsaturated compounds, especially for acrylic acid, methacrylic acid, acrylic esters and methacrylic esters.

Ethylenically unsaturated compounds are stored in permanently installed vessels that are suitable for the purpose, such as storage tanks (see for example Acrylic Acid, A Summary of Safety and Handling, 4th edition 2013, 7 Bulk Storage Facilities and Accessories). Preferably, the storage of the ethylenically unsaturated compounds in the respective vessels is preferably for at least one hour, more preferably at least 10 hours, most preferably at least 100 hours. Although the duration of storage under appropriate conditions is theoretically unlimited, the duration of storage is generally reduced to a minimum for economic reasons. Preferably, the storage of the ethylenically unsaturated compounds in the respective vessels is for not more than 180 days, more preferably for not more than 90 days, most preferably for not more than 30 days. Particularly preferred ranges result from the free combination of the aforementioned lower and upper ranges.

Ethylenically unsaturated compounds are typically transported in suitable transportable vessels, such as tanks or drums, by ship, rail and/or truck (see for example Acrylic Acid, A Summary of Safety and Handling, 4th edition 2013, 9 Safe Transport of Acrylic Acid). The transportable metals may of course also be permanently installed on the corresponding means of transportation, for example tanker ships or rail tank cars. It is of course also possible for vessels to be stored for a certain time in a particular place before they are transported to another site. Ethylenically unsaturated compounds may also be transported in pipelines or hoses, for example to a storage tank after purification of the ethylenically unsaturated compounds, in the case of transfer from the storage tank into a transportable vessel, and/or in the case of transfer from one transportable vessel into another. Preferably, the transportation of the ethylenically unsaturated compounds in the respective vessels is preferably for at least one hour, more preferably at least 10 hours, most preferably at least 100 hours. Although the duration under appropriate conditions is theoretically unlimited, the duration is generally reduced to a minimum for economic and safety reasons. Transportation of the ethylenically unsaturated compounds in the respective vessels is preferably for not more than 180 days, more preferably for not more than 90 days, most preferably for not more than 30 days. Particularly preferred ranges result from the free combination of the aforementioned lower and upper ranges.

EP 0 850 916 A1 discloses mixtures of polymerization inhibitors for basic monomers. The mixtures comprise phenols.

It was an object of the present invention to provide a method of storing and/or transporting ethylenically unsaturated compounds. The polymerization inhibitors are to ensure sufficient stabilization of the ethylenically unsaturated compounds against unwanted free-radical polymerization. In the intended free-radical polymerization, however, there shall be no need to separate the polymerization in from the mixture before the polymerization. The polymerization inhibitors are therefore not to act as chain transfer agents and/or polymerization inhibitors in the intended free-radical polymerization. In addition, the polymerization inhibitors were not to lead to unwanted discoloration in the course of polymerization.

The object is achieved by a method of storage and/or transport of an ethylenically unsaturated compound, where the ethylenically unsaturated compound has a purity of at least 90% by weight, the mass of stored and/or transported ethylenically unsaturated compound is at least 100 kg, the ethylenically unsaturated compound has a temperature during storage and/or transport of less than 50° C. and the ethylenically unsaturated compound is a carboxylic acid or a carboxylic ester, wherein the ethylenically unsaturated compound comprises from 0.0001% to 0.0750% by weight (1 to 750 ppm), based on the total amount of ethylenically unsaturated compound, of a compound of the general formula (I)

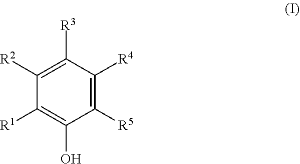

where
R¹, R³ and R⁵ is independently methyl or ethyl, preferably methyl, and
R² and R⁴ is independently H, methyl or ethyl, preferably H or methyl, more preferably H.

Suitable compounds of the general formula (I) are, for example, 2,4,6-trimethylphenol (TMP), 2-ethyl-4,6-dimethylphenol, 2,3,4,6-tetramethylphenol, 3-ethyl-2,4,6-trimethylphenol, 2-ethyl-3,4,6-trimethylphenol, 2,3-diethyl-4,6-dimethylphenol,
4-ethyl-2,6-dimethylphenol, 2,4-diethyl-6-methylphenol, 4-ethyl-2,3,6-trimethylphenol, 3,4-diethyl-2,6-dimethylphenol, 2,4-diethyl-3,6-dimethylphenol, 2,3,4-triethyl-6-methylphenol,
2,4,5,6-tetramethylphenol, 2-ethyl-4,5,6-trimethylphenol, 2,3,4,5,6-pentamethylphenol, 3-ethyl-2,4,5,6-tetramethylphenol, 2-ethyl-3,4,5,6-tetramethylphenol, 2,3-diethyl-4,5,6-trimethylphenol,
4-ethyl-2,5,6-trimethylphenol, 2,4-diethyl-5,6-dimethylphenol, 4-ethyl-2,3,5,6-tetramethylphenol, 3,4-diethyl-2,5,6-trimethylphenol, 2,4-diethyl-3,5,6-trimethylphenol, 2,3,4-triethyl-5,6-dimethylphenol,
5-ethyl-2,4,6-trimethylphenol, 2,5-diethyl-4,6-dimethylphenol, 5-ethyl-2,3,4,6-tetramethylphenol, 3,5-diethyl-2,4,6-trimethylphenol, 2,5-diethyl-3,4,6-trimethylphenol, 2,3,5-triethyl-4,6-dimethylphenol,
4,5-diethyl-2,6-dimethylphenol, 2,4,5-triethyl-6-methylphenol, 4,5-diethyl-2,3,6-trimethylphenol, 3,4,5-triethyl-2,6-dimethylphenol, 2,4,5-triethyl-3,6-dimethylphenol, 2,3,4,5-tetraethyl-6-methylphenol,
6-ethyl-2,4-dimethylphenol, 2,6-diethyl-6-methylphenol, 6-ethyl-2,3,4-trimethylphenol, 3,6-diethyl-2,4-dimethylphenol, 2,6-diethyl-3,4-dimethylphenol, 2,3,6-triethyl-4-methylphenol,
4,6-diethyl-2-methylphenol, 2,4,6-triethylphenol, 4,6-diethyl-2,3-dimethylphenol, 3,4,6-triethyl-2-methylphenol, 2,4,6-triethyl-3-methylphenol, 2,3,4,6-tetraethylphenol,
6-ethyl-2,4,5-trimethylphenol, 2,6-diethyl-4,5-dimethylphenol, 6-ethyl-2,3,4,5-tetramethylphenol, 3,6-diethyl-2,4,5-trimethylphenol, 2,6-diethyl-3,4,5-trimethylphenol, 2,3,6-triethyl-4,5-dimethylphenol,
4,6-diethyl-2,5-dimethylphenol, 2,4,6-triethyl-5-methylphenol, 4,6-diethyl-2,3,5-trimethylphenol, 3,4,6-triethyl-2,5-dimethylphenol, 2,4,6-triethyl-3,5-dimethylphenol, 2,3,4,6-tetraethyl-5-methylphenol,
5,6-diethyl-2,4-dimethylphenol, 2,5,6-triethyl-4-methylphenol, 5,6-diethyl-2,3,4-trimethylphenol, 3,5,6-triethyl-2,4-dimethylphenol, 2,5,6-triethyl-3,4-dimethylphenol, 2,3,5,6-tetraethyl-4-methylphenol,
4,5,6-triethyl-2-methylphenol, 2,4,5,6-tetraethylphenol, 4,5,6-triethyl-2,3-dimethylphenol, 3,4,5,6-tetraethyl-2-methylphenol, 2,4,5,6-tetraethyl-3-methylphenol and 2,3,4,5,6-pentaethylphenol.

The purity of the ethylenically unsaturated compound is preferably at least 95% by weight, more preferably at least 98% by weight, most preferably at least 99% by weight.

The mass of stored and/or transported ethylenically unsaturated compound is preferably at least 200 kg, more preferably at least 500 kg, most preferably at least 1000 kg. The mass of stored and/or transported ethylenically unsaturated compound is typically less than 10 000 000 kg.

The ethylenically unsaturated compound during storage and/or transport has a temperature of preferably less than 45° C., more preferably less than 40° C., most preferably less than 35° C. In the case of acrylic acid, the temperature during storage and/or transport should be at least 15° C.

The ethylenically unsaturated compound preferably comprises less than 0.0001% by weight each of a compound comprising amido groups, an ester of phosphorous acid, a phosphoric acid or a phosphine, based in each case on the total amount of ethylenically unsaturated compound.

In a preferred embodiment of the present invention, the compound of the general formula (I) is used essentially as the sole polymerization inhibitor.

The ethylenically unsaturated compound preferably comprises from 0.0002% to 0.0600% by weight (2 to 600 ppm), more preferably from 0.0005% to 0.0450% by weight (5 to 450 ppm), most preferably from 0.0010% to 0.0300% by weight (10 to 300 ppm) of a compound of the general formula (I), based in each case on the total amount of ethylenically unsaturated compound.

The ethylenically unsaturated compounds are preferably mono-, di- or triethylenically unsaturated C₃- to C₈-carboxylic acids, mono-, di- or triethylenically unsaturated C₃- to C₈-carboxylic esters having 1 to 20 carbon atoms in the ester groups.

Particular preference is given to mono-, di- or triethylenically unsaturated C₃- to C₆-carboxylic acids, for example acrylic acid, methacrylic acid, dimethacrylic acid, ethacrylic acid, citraconic acid, methylenemalonic acid, crotonic acid, fumaric acid, mesaconic acid, itaconic acid and maleic acid, mono-, di- or triethylenically unsaturated C₃- to C₆-carboxylic esters having 1 to 12 carbon atoms in the ester groups, for example acrylic esters with C₁- to C₁₂-alkyl, methacrylic esters with C₁- to C₁₂-alkyl, dimethacrylic esters with C₁- to C₁₂-alkyl, ethacrylic esters with C₁- to C₁₂-alkyl, citraconic esters with C₁- to C₁₂-alkyl, methylenemalonic esters with C₁- to C₁₂-alkyl, crotonic esters with C₁- to C₁₂-alkyl, fumaric esters with C₁- to C₁₂-alkyl, mesaconic esters with C₁- to C₁₂-alkyl, itaconic esters with C₁- to C₁₂-alkyl and maleic esters with C₁- to C₁₂-alkyl.

Particular preference is given to acrylic acid, methacrylic acid, acrylic esters with C₁ to C₈ alkyl, such as methyl acrylate, ethyl acrylate, n-butyl acrylate, and 2-ethylhexyl acrylate, and methacrylic esters with C₁ to C₈ alkyl, such as methyl methacrylate.

Further suitable ethylenically unsaturated compounds are dipropylene glycol diacrylate, tripropylene glycol diacrylate, polyethylene glycol diacrylate, glycerol triacrylate, ethoxylated glycerol triacrylate, trimethylolpropane triacrylate, ethoxylated trimethylolpropane triacrylate, butanediol monoacrylate, dicyclopentadienyl acrylate, 2-dimethylaminoethyl acrylate, 2-hydroxyethyl acrylate, and 2-hydroxypropyl acrylate.

With acrylic acid and/or methacrylic acid as the polymerizable compound, the total amount of compounds of the general formula (I) in the mixture is preferably from 0.0050% to 0.0600% by weight (50 to 600 ppm), more preferably from 0.0100% to 0.0450% by weight (100 to 450 ppm), even more preferably from 0.0150% to 0.0300% by weight (150 to 300 ppm), based in each case on the total amount of acrylic acid and/or methacrylic acid.

Acrylic acid and/or methacrylic acid are typically stored and/or transported in stainless steel vessels. Suitable stainless steel vessels comprise 16.5% to 19.5% by weight of chromium and from 8.0% to 13.5% by weight of nickel.

With methyl acrylate, methyl methacrylate, ethyl acrylate, n-butyl acrylate and/or 2-ethylhexyl acrylate as polymerizable compound, the total amount of compounds of the general formula (I) in the mixture is preferably from 0.0002% to 0.0060% by weight (2 to 60 ppm), more preferably from 0.0005% to 0.0040% by weight (5 to 40 ppm), even more preferably from 0.0010% to 0.0020% by weight (10 to 20 ppm), based in each case on the total amount of methyl acrylate, methyl methacrylate, ethyl acrylate, n-butyl acrylate and/or 2-ethylhexyl acrylate.

The ethylenically unsaturated compound is typically stored and/or transported in an oxygen-containing atmosphere.

The ethylenically unsaturated compound is preferably stored in a vessel in an oxygen-containing atmosphere having an oxygen content of 5% to 10% by volume and the mixture in the vessel is regularly recirculated, for example by pumped recirculation of the entirety of the tank contents at least once a week. The relatively low oxygen content prevents the formation of ignitable gas mixtures in the vessel. The recirculation replaces consumed dissolved oxygen in the liquid ethylenically unsaturated compound.

The present invention further provides a method of free-radical polymerization, wherein at least one of the above-described ethylenically unsaturated compounds is polymerized by means of a polymerization initiator, and for the use of one of the above-described ethylenically unsaturated compounds for free-radical polymerization by means of a polymerization initiator.

By the free-radical polymerization of the invention, for example, water-soluble and water-swellable polyacrylic acids and sodium salts thereof are obtainable.

Free-radical polymerization itself is common knowledge and is typically conducted in solution. Suitable polymerization initiators are all compounds that can break down to free radicals under the chosen reaction conditions, for example thermal initiators, redox initiators, photoinitiators. Suitable thermal initiators are peroxomono- and -disulfates, and peroxomono- and -diphosphates. Suitable redox initiators are sodium peroxodisulfate/ascorbic acid, hydrogen peroxide/ascorbic acid, sodium peroxodisulfate/sodium hypophosphite, sodium peroxodisulfate/sodium bisulfite and hydrogen peroxide/sodium bisulfite.

### EXAMPLES

### Inhibition of Polymerization Experiments

The ethylenically unsaturated compound used was distilled twice to remove the hydroquinone monomethyl ether (MEHQ) polymerization inhibitor. The stated amount of the specified polymerization inhibitor was added in each case to the ethylenically unsaturated compound. MEHQ and 2,4,6-trimethylphenol (TMP) were used.

0.5 ml of each mixture was transferred to a 1.8 ml ampoule and stored at the specified temperature in an air circulation oven.

In each test series, at least three ampoules of each mixture were filled and tested, with the average time for complete polymerization determined by visual examination.

TABLE 1
Mixtures comprising acrylic acid

| Polymerization | | | Time before |
|---|---|---|---|
| inhibitor | Amount | Temperature | polymerization |
| none*) | | 80° C. | 4.18 h |
| MEHQ*) | 0.0010% by wt. | 80° C. | 7.74 h |
| MEHQ*) | 0.0020% by wt. | 80° C. | 9.63 h |
| MEHQ*) | 0.0040% by wt. | 80° C. | 17.18 h |
| MEHQ*) | 0.0060% by wt. | 80° C. | 24.72 h |
| TMP | 0.0010% by wt. | 80° C. | 6.97 h |
| TMP | 0.0020% by wt. | 80° C. | 11.99 h |
| TMP | 0.0040% by wt. | 80° C. | 20.01 h |
| TMP | 0.0060% by wt. | 80° C. | 29.22 h |

*)comparative

TABLE 2
Mixtures comprising methacrylic acid

| Polymerization | | | Time before |
|---|---|---|---|
| inhibitor | Amount | Temperature | polymerization |
| none*) | | 80° C. | 4.76 h |
| MEHQ*) | 0.0010% by wt. | 80° C. | 9.81 h |
| MEHQ*) | 0.0020% by wt. | 80° C. | 17.75 h |
| MEHQ*) | 0.0040% by wt. | 80° C. | 27.39 h |
| MEHQ*) | 0.0060% by wt. | 80° C. | 35.79 h |
| TMP | 0.0010% by wt. | 80° C. | 11.28 h |
| TMP | 0.0020% by wt. | 80° C. | 15.49 h |
| TMP | 0.0040% by wt. | 80° C. | 24.18 h |
| TMP | 0.0060% by wt. | 80° C. | 32.48 h |

*)comparative

TABLE 3
Mixtures with methyl acrylate

| Polymerization | | | Time before |
|---|---|---|---|
| inhibitor | Amount | Temperature | polymerization |
| none*) | | 100° C. | 3.21 h |
| MEHQ*) | 0.0005% by wt. | 100° C. | 5.06 h |
| MEHQ*) | 0.0010% by wt. | 100° C. | 7.69 h |
| MEHQ*) | 0.0020% by wt. | 100° C. | 13.67 h |
| TMP | 0.0005% by wt. | 100° C. | 5.60 h |
| TMP | 0.0010% by wt. | 100° C. | 10.43 h |
| TMP | 0.0020% by wt. | 100° C. | 27.30 h |

*)comparative

TABLE 4
Mixtures with n-butyl acrylate

| Polymerization | | | Time before |
|---|---|---|---|
| inhibitor | Amount | Temperature | polymerization |
| none*) | | 100° C. | 1.14 h |
| MEHQ*) | 0.0005% by wt. | 100° C. | 2.63 h |
| MEHQ*) | 0.0010% by wt. | 100° C. | 4.48 h |
| MEHQ*) | 0.0020% by wt. | 100° C. | 10.54 h |
| TMP | 0.0005% by wt. | 100° C. | 2.69 h |
| TMP | 0.0010% by wt. | 100° C. | 5.57 h |
| TMP | 0.0020% by wt. | 100° C. | 15.97 h |

*)comparative

2,4,6-Trimethylphenol (TMP) has comparable to distinctly better inhibiting action versus MEHQ.

### Polymerization Experiments

### Polymerization of Acrylic Acid

A reaction vessel was initially charged under a nitrogen atmosphere with 450 g of water. The initially charged reaction mixture was heated to 95° C. with stirring. On attainment of a temperature of 95° C., three streams were metered in with stirring and while maintaining the temperature.
Stream 1:500 g of acrylic acid, metered in over 5 h
Stream 2:15 g of sodium hypophosphite in 35 g of deionized water, metered in over 4.75 h
Stream 3:5 g of sodium peroxodisulfate in 66.4 g of deionized water, metered in over 5.25 h

After addition of the three streams, the reaction mixture was stirred at 95° C. for a further hour. The reaction mixture was then cooled to room temperature and 80 g of water added.

The acrylic acid used in stream 1 was stabilized with MEHQ or 2,4,6-trimethylphenol (TMP).

The polymers obtained were analyzed by GPC (calibration with Na-PAA standard, eluent: 0.01 mol/l phosphate buffer pH 7.4 in distilled water containing 0.01 M NaN₃).

TABLE 5
Polymerization of acrylic acid

| Polymerization | | |
|---|---|---|
| inhibitor | Amount | Weight average Mw |
| MEHQ*) | 0.020% by wt. | 9400 g/mol |
| TMP | 0.020% by wt. | 9480 g/mol |

*)comparative

2,4,6-Trimethylphenol (TMP) with the concentration examined leads to polyacrylic acids having a comparable molar mass to that when the standard concentration of MEHQ is used.

### Polymerization of Sodium Acrylate or Sodium Methacrylate

Under a nitrogen atmosphere, a reaction vessel was charged with 52 g of deionized water and 60 g of aqueous sodium acrylate solution or aqueous sodium methacrylate solution (in each case 37.2% strength by weight and stabilized with 0.01% by weight of MeHQ or TMP).

Subsequently, 0.4 g of sodium peroxodisulfate dissolved in 10 g of deionized water was added, and the mixture was heated to 80° C. while stirring. The reaction mixture was stirred at 80° C. for 4 h and then cooled down to ambient temperature.

In all the experiments with MEHQ, the solution changed color during the heating (at about 60° C.).

TABLE 6
Polymerization of sodium acrylate or sodium methacrylate

| Polymerization inhibitor | Monomer | Color number |
|---|---|---|
| MEHQ*) | sodium acrylate | 360 |
| TMP | sodium acrylate | 51 |
| MEHQ*) | sodium | 132 |
| | methacrylate | |
| TMP | sodium | 25 |
| | methacrylate | |

*)comparative

Color numbers were measured to DIN EN ISO 6271 (2005).

2,4,6-Trimethylphenol (TMP) leads to a much lesser degree of unwanted discoloration than when MEHQ is used.

1.-15. (canceled)

16. A method of storage and/or transport of an ethylenically unsaturated compound, where the ethylenically unsaturated compound has a purity of at least 90% by weight, the mass of stored and/or transported ethylenically unsaturated compound is at least 100 kg, the ethylenically unsaturated compound has a temperature during storage and/or transport of less than 50° C. and the ethylenically unsaturated compound is a carboxylic acid or a carboxylic ester, wherein the ethylenically unsaturated compound comprises from 0.0001% to 0.0750% by weight, based on the total amount of ethylenically unsaturated compound, of a compound of the general formula (I) 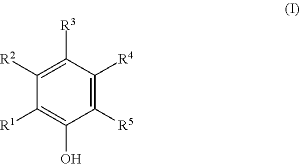
where
R¹, R³ and R⁵ is independently methyl or ethyl and
R² and R⁴ is independently H, methyl or ethyl.

17. The method according to claim 16, wherein the ethylenically unsaturated compound comprises less than 0.0001% by weight each of a compound comprising amido groups, an ester of phosphorous acid, a phosphoric acid or a phosphine, based in each case on the total amount of ethylenically unsaturated compound.

18. The method according to claim 16, wherein the compound of the general formula (I) is used essentially as the sole polymerization inhibitor.

19. The method according to claim 16, wherein 2,4,6-trimethylphenol is used as compound of the general formula (I).

20. The method according to claim 16, wherein storage and/or transport of the ethylenically unsaturated compound extends over a period of at least 10 hours.

21. The method according to claim 16, wherein the ethylenically unsaturated compound is acrylic acid and/or methacrylic acid.

22. The method according to claim 21, wherein the ethylenically unsaturated compound comprises from 0.0050% to 0.0600% by weight, based on the total amount of ethylenically unsaturated compound, of a compound of the general formula (I).

23. The method according to claim 21, wherein the ethylenically unsaturated compound comprises from 0.0150% to 0.0300% by weight, based on the total amount of ethylenically unsaturated compound, of a compound of the general formula (I).

24. The method according to claim 16, wherein the ethylenically unsaturated compound is methyl acrylate, methyl methacrylate, ethyl acrylate, n-butyl acrylate and/or 2-ethylhexyl acrylate.

25. The method according to claim 24, wherein the ethylenically unsaturated compound comprises from 0.0002% to 0.0060% by weight, based on the total amount of ethylenically unsaturated compound, of a compound of the general formula (I).

26. The method according to claim 24, wherein the ethylenically unsaturated compound comprises from 0.0010% to 0.0020% by weight, based on the total amount of ethylenically unsaturated compound, of a compound of the general formula (I).

27. The method according to claim 16, wherein the ethylenically unsaturated compound is stored and/or transported under an oxygenous atmosphere.

28. The method according to claim 16, wherein the ethylenically unsaturated compound is stored in a vessel under an oxygen-containing atmosphere having an oxygen content of 5% to 10% by volume and the ethylenically unsaturated compound in the vessel is regularly recirculated.

29. A method of free-radical polymerization, where at least one ethylenically unsaturated compound is polymerized by means of a polymerization initiator and the ethylenically unsaturated compound is a carboxylic acid or a carboxylic ester, wherein the ethylenically unsaturated compound used has a purity of at least 90% by weight and the ethylenically unsaturated compound comprises from 0.0001% to 0.0750% by weight, based on the total amount of ethylenically unsaturated compound, of a compound of the general formula (I) 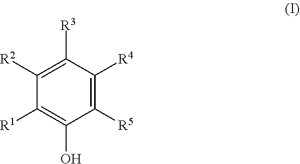
where
R¹, R³ and R⁵ is independently methyl or ethyl and
R² and R⁴ is independently H, methyl or ethyl.

30. A use of an ethylenically unsaturated carbonyl compound for free-radical polymerization by means of a polymerization initiator, wherein the ethylenically unsaturated compound is a carboxylic acid or a carboxylic ester, the ethylenically unsaturated compound has a purity of at least 90% by weight and the ethylenically unsaturated compound comprises from 0.0001% to 0.0750% by weight, based on the total amount of ethylenically unsaturated compound, of a compound of the general formula (I) 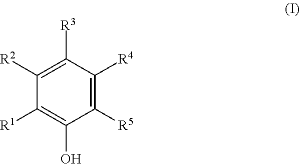
where
R¹, R³ and R⁵ is independently methyl or ethyl and
R² and R⁴ is independently H, methyl or ethyl.
